(19) United States
(12) Patent Application Publication
(10) Pub. No.: US 2014/0370531 A1
(43) Pub. Date: Dec. 18, 2014

(54) METHOD OF DIAGNOSING MILD TRAUMATIC BRAIN INJURY

(71) Applicants: Brian Blyth, Fairport, NY (US); Jeffrey Bazarian, Honeoye Falls, NY (US)

(72) Inventors: Brian Blyth, Fairport, NY (US); Jeffrey Bazarian, Honeoye Falls, NY (US)

(73) Assignee: UNIVERSITY OF ROCHESTER, Rochester, NY (US)

(21) Appl. No.: 14/365,550

(22) PCT Filed: Dec. 11, 2012
(86) PCT No.: PCT/US12/69002

§ 371 Date: Jun. 13, 2014

Related U.S. Application Data
(60) Provisional application No. 61/570,697, filed on Dec. 14, 2011.

(51) Int. Cl. G01N 33/68

(52) U.S. Cl. CPC G01N 33/6896; G01N 2333/775
USPC 435/7.92

(57) ABSTRACT

The present invention relates to a method of determining whether a subject has suffered a mild traumatic brain injury. The method comprises selecting a subject exposed to a head trauma and determining an apoA-1 level in a body fluid sample obtained from the selected subject, wherein a decreased apoA-1 level in the body fluid sample indicates that the subject has suffered a mild traumatic brain injury. The invention also relates to a kit for determining whether a subject has suffered a mild traumatic brain injury.

This application claim the priority benefit of U.S. Provisional Patent Application Ser. No. 61/570,697, filed Dec. 14, 2011, which is hereby incorporated by reference in its entirety.

This invention was made with government support under National Institutes of Health U.S. Public Health Service Grants K23 NS41952 and R01 HD051865. The government has certain rights in the invention.

### FIELD OF THE INVENTION

This invention relates to a diagnostic method of determining whether a subject has suffered mild traumatic brain injury by measuring apolipoprotein A-1 (apoA-1).

### BACKGROUND OF THE INVENTION

Mild traumatic brain injury (mTBI) affects 1.7 million patients annually in the United States (Faul et al., “Traumatic Brain Injury in the United States: Emergency Department Visits, Hospitalizations and Deaths 2002-2006,” Centers for Disease Control and Prevention, National Center for Injury Prevention and Control, Atlanta, Ga. (2010)) and is a significant risk factor for the development of neurodegenerative illness (Nemetz et al., “Traumatic Brain Injury and Time to Onset of Alzheimer's Disease: A Population-Based Study,” Am. J. Epidemiol. 149:32-40 (1999)). Diagnosis is made subjectively because there are no consistent objective findings associated with mTBI (THE CONCUSSION/MTBI WORKING GROUP, VA/DOD CLINICAL PRACTICE GUIDELINE FOR MANAGEMENT OF CONCUSSION/MILD TRAUMATIC BRAIN INJURY (2009)). Inaccurate diagnosis is common Patients suffering mTBI due to combat or sports often deliberately under report their symptoms to avoid being separated from their team members. Because mTBI frequently results in subtle acute cognitive deficits, these patients are at risk for further injury. An intense search for accurate and clinically useful molecular biomarkers has largely failed, likely due to the presence of the blood brain barrier (BBB) which prevents passage of most molecules from brain into the peripheral circulation (Morganti-Kossmann et al., “TGF-beta is Elevated in the CSF of Patients with Severe Traumatic Brain Injuries and Parallels Blood-Brain Barrier Function,” J. Neurotrauma 16:617-628 (1999); Blyth et al., “Validation of Serum Markers for Blood-Brain Barrier Disruption in Traumatic Brain Injury,” J. Neurotrauma. 26:1497-1507 (2009)).

TBI has been called the “signature injury” of the current conflicts in Iraq and Afghanistan. Nearly 90% of these injuries are classified as mild or a concussion. Acutely and sub-acutely, mTBI often leads to subtle cognitive dysfunction. This post-mTBI cognitive dysfunction is particularly problematic in military populations where the injured subject is often making decisions for himself or a group that have life and death consequences. Diagnosis of mTBI is based on a clinical history alone. Reliable objective aids for the diagnosis of mTBI are not available. Military personnel in combat situations are often unwilling to provide an accurate history after mTBI because they do not want to abandon their units or, conversely, they wish to avoid further combat. Thus, there is a need for a reliable and objective test for the diagnosis of mTBI. Such a test would have substantial utility both in military populations in war zones as well as in civilians presenting to emergency departments. Additionally, an objective test would be useful in outpatient populations to identify the need for further hospital-based diagnosis and treatment.

The present invention overcomes these and other deficiencies in the art.

### SUMMARY OF THE INVENTION

One aspect of the present invention relates to a method of determining whether a subject has suffered a mild traumatic brain injury. The method includes selecting a subject exposed to a head trauma, and determining an apoA-1 level in a body fluid sample obtained from the selected subject, wherein a decreased apoA-1 level in the body fluid sample (relative to control) indicates that the subject has suffered a mild traumatic brain injury.

A second aspect also relates to a method of diagnosing a subject for a mild traumatic brain injury. The method includes obtaining a body fluid sample from a subject exposed to a head trauma; and reacting the body fluid sample, or a portion thereof, with a reagent that binds specifically to apoA-1, and measuring the reagent/apoA-1 reaction product to determine an apoA-1 level in the obtained body fluid sample, wherein a decreased apoA-1 level in the body fluid sample indicates that the subject has suffered a mild traumatic brain injury.

A third aspect of the present invention relates to a method of treating an individual that has suffered a mild traumatic brain injury. The method includes: selecting a subject exposed to a head trauma; determining an apoA-1 level in a body fluid sample obtained from the selected subject, wherein a decreased apoA-1 level in the body fluid sample indicates that the subject has suffered a mild traumatic brain injury; and treating the subject having suffered a mild traumatic brain injury as determined by said determining.

Current diagnosis of mTBI requires an accurate clinical history. Confounding factors such as conditions mimicking TBI like syncope, intoxication, or seizures complicate clinical diagnosis of mTBI. Willful misreporting of symptoms is also a significant problem particularly with athletes and military personnel. For example, military personnel in Iraq and Afghanistan conflicts often rehearse answers to the standard field screening test for concussion in order to successfully answer in the event they are impaired following a concussion. The present invention circumvents these problems through application of an objective test.

Results of the present application demonstrate that serum concentrations of apoA-1 are decreased within six hours of mTBI. This elevation is an accurate surrogate for the clinical diagnosis of mTBI, as the decrease is specific to brain injury. These results demonstrate an adaptive mechanism in response to mTBI that is used in the present invention as a biomarker that obviates the influence of the BBB. Thus, decreased apoA-1 is a biomarker that is useful as an objective test for the diagnosis of mTBI. While S100B is a less accurate test for diagnosis of mTBI, it continues to be valuable to identify patients at high risk for traumatic injuries detectable with cranial computed tomography, and particularly when used in combination with apoA-1 levels.

### BRIEF DESCRIPTION OF THE DRAWINGS

FIG. 1 is a graph illustrating the mean-IQR (min-max) apoA-1 and S100B concentrations in two groups of subjects, control subjects and subjects having a head injury and meeting the criteria for inclusion.

FIG. 2 is a graph illustrating the mean apoA-1 and S100B concentrations in the two groups of subjects. Standard deviations are illustrated.

FIG. 3 is a graph illustrating mean apoA-1 and S100B concentrations in the two groups of subjects.

FIGS. 4A-B are Receiver Operator Characteristic (ROC) curve analyses of S100B and apoA1, respectively, in mTBI subjects versus controls.

FIG. 5 is graph illustrating the median apoA1 with IQR with blood brain barrier closed (S100B negative patients) versus open (S100B positive patients).

FIG. 6 is a graph illustrating the subject classification as mTBI-positive based on S100B alone.

FIG. 7 is a graph illustrating the subject classification as mTBI-positive based on both apoA-1 and S100B. A highly frequency of correctly diagnosed patients can be obtained when using both apoA-1 and S100B as diagnostic markers.

FIG. 8 is graph comparing the efficacy of using S100B alone, apoA-1 alone, and a combination of S100B and apoA-1 to diagnose mTBI patients. The combination of S100B and apoA-1 was much more efficient in diagnosing patients.

FIG. 9 illustrates ROC curves comparing S100B (red or lowest), apoA-1 (black or middle), and the combined test (green or uppermost). The area under the receiver operator characteristic curve for the combined test is 0.74.

### DETAILED DESCRIPTION OF THE INVENTION

The present invention relates to methods of determining whether a subject has suffered a mild traumatic brain injury (mTBI). The methods include selecting a subject exposed to a head trauma, and then determining the presence of or level of a particular biomarker in a body fluid sample obtained from the selected subject. Presence of the biomarker or decreased levels of the biomarker in the body fluid sample, relative to a standard or control, indicates that the subject has suffered mTBI.

A subject exposed to a head trauma includes any mammal, preferably human, that is conscious or unconscious but not comatose. The subject who is exposed to the head trauma may exhibit extra-cranial injuries or may exhibit no extra-cranial injuries.

The method of the present invention can be practiced on patients whose head trauma is produced, at least in part, by brain injuries including those produced by blunt head trauma or missile penetration.

Conscious, as used herein, has the conventional meaning, as set forth in Plum, et al., The Diagnosis of Stupor and Coma, CNS Series, Philadelphia:Davis (1982), which is hereby incorporated by reference. Conscious patients include those who have a capacity for reliable, reproducible, interactive behavior evidencing awareness of self or the environment. Conscious patients include patients who recover consciousness with less severe brain injury but who, because of their impaired cognitive function, do not reach independent living. Conscious patients do not include those who exhibit wakefulness but lack interaction (e.g., those deemed to be in a persistent vegetative state).

The selected subject who is conscious after exposure to a head trauma may be asymptomatic of any visible symptoms of traumatic brain injury. Conversely, the selected subject may exhibit various symptoms of brain injury and cognitive dysfunction.

This is in contrast to a subject who is unconscious at the time of the obtaining, as indicated by conditions such as a concussion or intracranial hemorrhage (e.g. intra-axial hematoma, epidural hematoma, and subdural hematoma).

As indicated above, the subject exposed to head trauma may exhibit extra-cranial injuries. Exemplary extra-cranial injuries include open head injuries, such as a visible assault to the head. Extra-cranial injuries may result from a gunshot wound, an accident or an object going through the skull into the brain (“missile injury to the brain”). This type of brain injury is likely to damage a specific area of the brain.

Alternatively, the subject exposed to a head trauma may exhibit only superficial external injuries or no extra-cranial injuries. In this instance, the subject may have no visible injury (e.g. a closed head injury), or may exhibit those symptoms by deficits in attention, intention, working memory, and/or awareness as described herein. mTBI may also include or result in any one, or more, of the following: cognition impairment; language impairment; conduct disorder; motor disorder; and any other neurological dysfunction. mTBI may occur with no loss of consciousness and possibly only a dazed feeling or confused state lasting a short time.

A brain injury may occur when there is a blow to the head as in a motor vehicle accident or a fall. In this case, the skull hits a stationary object and the brain, which is inside the skull, turns and twists on its axis (the brain stem), causing localized or widespread damage. Also, the brain, a soft mass surrounded by fluid that allows it to “float,” may rebound against the skull resulting in further damage.

In response to the head trauma, changes occur in the brain, which require monitoring to prevent further damage. The brain's size frequently increases after a severe head injury. This is called brain swelling and occurs when there is an increase in the amount of blood to the brain. Later in the illness, water may collect in the brain, which is called brain edema. Both brain swelling and brain edema result in excessive pressure in the brain called intracranial pressure (“ICP”).

Even mTBI may result in persisting debility, such as post-traumatic epilepsy, persistent vegetative state, or post-traumatic dementia in the absence of proper treatment. Other complications and late effects of brain injury include, but are not limited to, coma, meningitis, post-traumatic epilepsy, post-traumatic dementia, degeneration of nerve fibers, post-traumatic syringomyelia, or hemorrhage, for example. Although medical care administered may be minimal in the context of mTBI, persons with brain injury without coma may experience symptoms and impairments similar to those suffered by the survivor of a severe brain injury.

As used herein, the term “sample” in the context of the present invention is a body fluid sample. The body fluid sample can be any sample containing apoA-1. Of particular interest are samples that are serum, plasma, and whole blood. Those skilled in the art will recognize that plasma or whole blood, or a sub-fraction of whole blood, may be used. The body fluid sample may also be saliva, urine, sweat, cerebrospinal fluid, ascites, thoracic fluid, interstitial fluid, tears, bile, and mucous. These various body fluid samples may be obtained using standard procedures for the recovery of the particular body fluid.

For example, a blood or serum sample may be obtained by use of a standard blood draw, as disclosed in U.S. Pat. No. 4,263,922, which is hereby incorporated by reference in its entirety. Generally, in a standard blood draw, blood is drawn through a needle assembly and handle system into a collection tube. Subsequent to the blood draw, the needle assembly and the handle are removed from an end of the tube and a separate cap is fitted over each end of the tube to retain the blood sample in the tube for analysis. In the case of humans, a finger prick with a lancet or a blood draw via standard venipuncture are also convenient methods to obtain a body fluid sample.

Upon obtaining a blood sample from an individual who has suffered a head trauma, the drawn blood is preferably exposed immediately to an anticoagulant to preclude coagulation thereof. Known anticoagulants include without limitation heparin, EDTA, D-Phe-Pro-Arg chloromethyl ketone dihydrochloride (“PPACK”), and sodium citrate.

The body fluid sample may be obtained prior to determining whether the selected subject has undergone a head trauma. This may be useful in instances where there are no witnesses to the head trauma incident that inflicted the potential mTBI to the subject.

The determination of whether the subject has suffered mTBI can be completed immediately following exposure to head trauma, or at any time thereafter. The determination may be used as a method to determine follow up treatment, by testing the selected subject's apoA-1 levels at various time points during and after treatment for a previous head trauma. Ideally, determination of mTBI injury is completed by obtaining body fluid samples as soon as possible or immediately after exposure to head trauma, e.g. within the first hour after the injury. The body fluid sample may be obtained from the subject up to 24 hours after the trauma, preferably within about six hours after the trauma occurs. Additional body fluid samples may be further obtained within hours, days, or weeks after exposure to a head trauma.

In accordance with one emobdiment, the biomarker used to determine whether a subject has suffered an mTBI is the apolipoprotein apoA-1. Apolipoproteins are lipid-free components of the plasma lipoproteins obtained by treating isolated intact lipoproteins with organic solvents, detergents, or agents. ApoA-1 is a 28 kDa apolipoprotein that is primarily synthesized in the liver and small intestine. An exemplary apoA-1 amino acid sequence is provided at Genbank accession NM-000039, which is hereby incorporated by reference in its entirety. The physiochemical properties of apoA-1 allow it to associate with lipids and other proteins. As described herein, the present invention measures apoA-1 levels in a body fluid sample of a subject exposed to a head trauma.

ApoA-1 is catabolized in both the kidneys and the liver, with more than half being catabolized by the liver (Radar, “Molecular Regulation of HDL Metabolism and Function: Implications for Novel Therapies,” J. Clin. Invest. 116(12):3090-3100 (2006), Lewis et al., “New Insights Into the Regulation of HDL Metabolism and Reverse Cholesterol Transport,” Cir. Res. 96:1221-1232 (2005), both of which are hereby incorporated by reference in their entirety). As a major protein component of HDL in plasma, apoA-1 promotes cholesterol efflux from tissues to the liver for excretion. ApoA-1 is a cofactor for lecithin cholesterol acyl transferase (LCAT). ApoA-1 recruits additional phospholipids and free cholesterol via the ABCA1 pathway, forming nascent HDL. Nascent HDL acquires more lipid from other peripheral tissues and lipoproteins. LCAT, also known as phosphatidylcholine-sterol O-acyltransferase, is an enzyme that converts free cholesterol into cholesteryl ester (a more hydrophobic form of cholesterol). Since unesterified cholesterol migrates freely between lipoproteins it must be trapped by esterification to maintain its association with HDL. Once free cholesterol is converted into cholesteryl ester, it is then sequestered into the core of a lipoprotein particle, eventually making the newly synthesized HDL particle spherical and forcing the reaction to become unidirectional since the particles are removed from the surface. The enzyme is bound to HDLs and low-density lipoproteins in the plasma. Radar, “Molecular Regulation of HDL Metabolism and Function: Implications for Novel Therapies,” J. Clin. Invest. 116(12):3090-3100 (2006), Carter, “Convergence of Genes Implicated in Alzheimer's Disease on the Cerebral Cholesterol Shuttle: APP, Cholesterol, Lipoproteins, and Atherosclerosis,” Neurochem. Int. 50:12-38 (2007), both of which are hereby incorporated by reference in their entirety.

More is understood regarding the mechanisms of apoA-1 catabolism by the kidney than by the liver. Lipid-poor apoA-1 can be filtered at the level of the glomerulus and then catabolized by proximal renal tubular epithelial cells (Radar, “Molecular Regulation of HDL Metabolism and Function: Implications for Novel Therapies,” J. Clin. Invest. 116(12):3090-3100 (2006), which is hereby incorporated by reference in its entirety). Cubilin is an extracellular protein synthesized by proximal renal tubular cells and localized to the apical surface anchored by another protein called “amnionless” (Moestrup et al., “The Role of the Kidney in Lipid Metabolism,” Curr. Opin. Lipidol. 16:301-306 (2005), which is hereby incorporated by reference in its entirety). Cubilin binds HDL and apoA-1 with high affinity (Hammad et al., “Cubilin, the Endocytic Receptor for Intrinsic Factor-Vitamin B12 Complex, Mediates High-Density Lipoprotein Holoparticle Endocytosis,” Proc. Natl. Acad. Sci. U.S.A. 96:10158-10163 (1999); Kozyraki et al., “The Intrinsic Factor-Vitamin B12 Receptor, Cubilin, is a High-Affinity Apolipoprotein A-1 Receptor Facilitating Endocytosis of High-Density Lipoprotein,” Nat. Med. 5:656-661 (1999), which are hereby incorporated by reference in their entirety) and then interacts with a coreceptor called megalin, a member of the low-density lipoprotein receptor (LDLR) gene family, to mediate uptake and degradation of apoA-1 (Hammad et al., “Megalin Acts in Concert with Cubilin to Mediate Endocytosis of High Density Lipoproteins,” J. Biol. Chem. 275:12003-12008 (2000), which is hereby incorporated by reference in its entirety). However, functional cubilin deficiency in animals or humans is not associated with changes in plasma HDL-C or apoA-1 concentrations (Christensen et al., “Protein Reabsorption in Renal Proximal Tubule-Function and Dysfunction in Kidney Pathophysiology,” Pediatr. Nephroi. 19:714-721 (2004), which is hereby incorporated by reference in its entirety). Thus, it is doubtful that this renal tubular pathway is responsible for variation in HDL-C levels in humans.

The liver is also responsible for substantial degradation of apoA-1 (Glass et al., “Dissociation of Tissue Uptake of Cholesterol Ester From That of Apoprotein A-1 of Rat Plasma High Density Lipoprotein: Selective Delivery of Cholesterol Ester to Liver, Adrenal, and Gonad,” Proc. Natl. Acad. Sci. U.S.A. 80:5435-5439 (1983), which is hereby incorporated by reference in its entirety). The mechanisms of hepatic uptake and HDL apolipoprotein degradation remain poorly understood. One mechanism that likely plays at least some role is related to apoE (Radar, “Molecular Regulation of HDL Metabolism and Function: Implications for Novel Therapies,” J. Clin. Invest. 116(12):3090-3100 (2006), which is hereby incorporated by reference in its entirety).

ApoA-1 levels are decreased in individuals that have suffered an mTBI. As used herein, the term “decreased” is intended to mean that the measured apoA-1 levels in the obtained body fluid sample are lower than either a predicted normal range for non-mTBI subjects, a threshold apoA-1 level, or a control sample (e.g., one or more calibrations standards) measured simultaneously or previously. The control sample can be from the same subject at a time prior to the head trauma, from a different subject or panel of subjects not exposed to recent head trauma, or one or more calibration samples each containing a known quantity of apoA-1. The present invention identifies the presence of decreased apoA-1 levels compared to control or normal range apoA-1 levels as a means to detect mTBI following exposure to a head trauma. As demonstrated in the accompanying examples, average apoA-1 levels in subjects that have not undergone exposure to head trauma causing mTBI are higher than those subjects who experience mTBI.

One example of a suitable threshold (or cut-off) is 0.68 mg/ml. Comparable specificity but reduced sensitivity can be obtained using an ApoA-1 threshold level less than 0.68 mg/ml. Examples of threshold ApoA-1 levels that can be selected for comparable specificity include, without limitation, 0.60 mg/ml, 0.50 mg/ml, 0.40 mg/ml, 0.35 mg/ml, 0.30 mg/ml, 0.25 mg/ml, 0.20 mg/ml, and 0.15 mg/ml. Thresholds higher than 0.50 mg/ml can be selected, for example, between 0.60 and 0.80 mg/ml, or 0.68 mg/ml, but these thresholds have lower specificity and only slightly improved sensitivity. Using a higher threshold may warrant consideration of other indicia of mTBI or further monitoring prior to diagnosing mTBI.

When using a control sample from the same subject, a decrease in Apo-A1 level of about 5% or more, 10% or more, 20% or more, 25% or more, preferably 33% or more, constitutes a decreased Apo-A1 level. Substantially higher percentage decreases may warrant immediate diagnosis of mTBI, whereas lower percentage decreases in apoA-1 levels may warrant consideration of other indicia of mTBI or further monitoring prior to diagnosing mTBI.

The concentration of the apoA-1 biomarker may be measured by using standard immunodiagnostic techniques, including immunoassays such as competition, direct reaction, or sandwich type assays. Such assays include, but are not limited to, Western blots, agglutination tests, enzyme-labeled and mediated immunoassays such as ELISAs, biotin/avidin type assays, radioimmunoassay, immunoelectrophoresis, immunoprecipitation, gas chromatography, high performance liquid chromatography (HPLC), size exclusion chromatography, solid-phase affinity, etc. The concentration of the apoA-1 biomarker may also be measured by any assay type applied in the field of diagnostics, including but not restricted to assay methods based on enzymatic reactions, luminescence, in particular fluorescence or radio chemicals. The preferred detection methods comprise rapid test formats including immunochromatography (e.g., strip formats), radioimmunoassays, chemiluminescence- and fluorescence-immunoassays, immunoblot assays, enzyme-linked immunoassays (ELISA), luminex-based bead arrays, and protein microarray assays. See U.S. Pat. Publ. No. 20110086831 to Bergmann et al., which is hereby incorporated by reference in its entirety. The assay types can further be microtitre plate-based, chip-based, bead-based, wherein the biomarker proteins can be attached to the surface or in solution. The assays can be homogenous or heterogeneous assays, sandwich assays, competitive and non-competitive assays (THE IMMUNOASSAYHANDBOOK, Ed. David Wild, Elsevier LTD, Oxford; 3rd ed. (May 2005); Hultschig et al., “Recent Advances of Protein Microarrays,” Curr. Opin. Chem. Biol. 10(1):4-10 (2006), both of which are hereby incorporated by reference in their entirety).

Regardless of the embodiment, the assay includes a reagent, preferably though not exclusively an immunological reagent such as an antibody, antibody fragment, or antibody mimic, which reacts specifically with ApoA-1. The reaction product of ApoA-1 and the reagent can be detected in accordance with the assay protocol.

In one embodiment, the assay is in the form of a sandwich assay, which is a non-competitive immunoassay, wherein the ApoA1 is bound to a first antibody and to a second antibody. The first antibody may be bound to a solid phase, e.g., a bead, a surface of a well or other container, a chip or a strip, and the second antibody is an antibody which is labeled, e.g., with a dye, with a radioisotope, or a reactive or catalytically active moiety. The amount of labeled antibody bound to the solid phase is then measured by a method suitable for the label employed. The general composition and procedures involved with “sandwich assays” are well-established and known to the skilled person (THE IMMUNOASSAY HANDBOOK, Ed. David Wild, Elsevier LTD, Oxford; 3rd ed. (May 2005); Hultschig et al., “Recent Advances of Protein Microarrays,” Curr. Opin. Chem. Biol. 10(1):4-10 (2006), both of which are hereby incorporated by reference in their entirety).

In another embodiment the assay comprises two capture molecules, preferably antibodies, which are both present as dispersions in a liquid reaction mixture, wherein a first labeling component is attached to the first capture molecule, wherein the first labeling component is part of a labeling system based on fluorescence- or chemiluminescence-quenching or amplification, and a second labeling component of the marking system is attached to the second capture molecule, so that upon binding of both capture molecules to the analyte a measurable signal is generated that allows for the detection of the formed ApoA-1/sandwich complexes in the solution. See U.S. Pat. Publ. No. 20110086831 to Bergmann et al., which is hereby incorporated by reference in its entirety.

In another embodiment, the labeling system includes rare earth cryptates or rare earth chelates in combination with fluorescence dye or chemiluminescence dye, in particular a dye of the cyanine type. See U.S. Pat. Publ. No. 20110086831 to Bergmann et al., which is hereby incorporated by reference in its entirety.

In the context of the present invention, fluorescence based assays comprise the use of dyes, which may for instance be selected from the group comprising FAM (5- or 6-carboxyfluorescein), VIC, NED, Fluorescein, Fluorescein-isothiocyanate (FITC), IRD-700/800, Cyanine dyes such as CY3, CY5, CY3.5, CY5.5, Cy7, Xanthen, 6-Carboxy-2′,4′,7′,4,7-hexachlorofluorescein (HEX), TET, 6-Carboxy-4′,5′-dichloro-2′,7′-dimethoxy-fluorescein (JOE), N,N,N′,N′-Tetramethyl-6-carboxyrhodamine (TAMRA), 6-Carboxy-X-rhodamine (ROX), 5-Carboxyrhodamine-6G (R6G5), 6-carboxyrhodamine-6G (RG6), Rhodamine, Rhodamine Green, Rhodamine Red, Rhodamine 110, BODIPY dyes, such as BODIPY TMR, Oregon Green, Coumarines such as Umbelliferone, Benzimides such as Hoechst 33258; Phenanthridines such as Texas Red, Yakima Yellow, Alexa Fluor, PET, Ethidiumbromide, Acridinium dyes, Carbazol dyes, Phenoxazine dyes, Porphyrine dyes, Polymethin dyes, and the like. In the context of the present invention, chemiluminescence based assays comprise the use of dyes, based on the physical principles described for chemiluminescent materials in KIRK-OTHMER, ENCYCLOPEDIA OF CHEMICAL TECHNOLOGY, 4th ed., exec. Ed., J. I. Kroschwitz; Editor, M. Howe-Grant, John Wiley & Sons, 15:518-562 (1993), which is hereby incorporated by reference in its entirety.

Lateral flow tests based on the principles of chromatographic immunoassay may also be used in the present invention. Lateral flow tests use strips coated with antibodies and/or other reactants that, upon reaction with ApoA-1 present in a contacted blood specimen or other sample, result in the appearance of colored lines (i.e., an indicator) indicative of the presence in a patient of ApoA-1 above a certain threshold value, which triggered the formation of ApoA-1/antibody complexes. The general format of the strip exhibits a single test line, so-called “T-line” and a single control line, so-called “C-line”. The control line is used as an indicator of functional validity. More recent test strips are offered that group multiple test lines for the detection of more than one kind of substance in the contacted sample. See U.S. Pat. Publ. No. US20040191760 to Zhou et al., which is hereby incorporated by reference in its entirety.

Depending upon the assay and detection methods used, antibodies for use in the present invention may optionally be conjugated to other proteins or chemical markers, to facilitate detection of the antibody binding to ApoA-1. Preferred are antibodies conjugated to enzymatic elements such as alkaline phosphatase, or readily detectable groups such as colloidal gold, biotin, and streptavidin. Other suitable conjugation agents are well known in the art. See U.S. Pat. Publ. No. 20040115748 to Kelley, which is hereby incorporated by reference in its entirety.

In particular embodiments, these detection methods involve contacting the obtained sample with a binding partner capable of selectively interacting with ApoA-1 biomarker. In one embodiment, the binding partner is an anti-apoA-1 antibody, which refers to an antibody or a binding fragment thereof which recognizes ApoA-1. The anti-apoA-1 antibody can be polyclonal or monoclonal, preferably monoclonal, as well as any fragments thereof that bind specifically to ApoA-1. In another embodiment, the binding partner is an antibody mimic, which can be a nucleic acid or peptide aptamer, or a polypeptide scaffold containing one or more variable regions that bind specifically to ApoA-1.

Polyclonal antibodies and fragments thereof can be raised according to known methods by administering the appropriate antigen or epitope to a host animal selected, e.g., from pigs, cows, horses, rabbits, goats, sheep, and mice, among others, and then recovering serum (containing the antibodies) from the host animal Various adjuvants known in the art can be used to enhance antibody production. Although antibodies useful in practicing the invention can be polyclonal, monoclonal antibodies are preferred.

Monoclonal antibodies can be prepared using hybridoma methods, such as those described by Kohler and Milstein, “Continuous Cultures of Fused Cells Secreting Antibody of Predefined Specificity,” Nature 256:495-7 (1975), which is hereby incorporated by reference in its entirety. Using the hybridoma method, a host animal is immunized to elicit the production by lymphocytes of antibodies that will specifically bind to an immunizing antigen. Alternatively, lymphocytes can be immunized in vitro. Following immunization, the lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against the scaffold, as determined by immunoprecipitation, immunoblotting, or by an in vitro binding assay such as radioimmunoassay (RIA) or enzyme-linked immunosorbent assay (ELISA) can then be propagated either in in vitro culture using standard methods (JAMES W. GODING, MONOCLONAL ANTIBODIES: PRINCIPLES AND PRACTICE (Academic Press 1986), which is hereby incorporated by reference in its entirety) or in vivo as ascites tumors in an animal The monoclonal antibodies can then be purified from the culture medium or ascites fluid as described for polyclonal antibodies above.

Alternatively monoclonal antibodies can also be made using recombinant DNA methods as described in U.S. Pat. No. 4,816,567 to Cabilly et al., which is hereby incorporated by reference in its entirety. Polynucleotides encoding a monoclonal antibody are isolated from mature B-cells or hybridoma cells by RT-PCR using oligonucleotide primers that specifically amplify the genes encoding the heavy and light chains of the antibody. The isolated polynucleotides encoding the heavy and light chains are then cloned into suitable expression vectors, which when transfected into host cells, monoclonal antibodies are generated by the host cells. Also, recombinant monoclonal antibodies or fragments thereof of the desired species can be isolated from phage display libraries as described (McCafferty et al., “Phage Antibodies: Filamentous Phage Displaying Antibody Variable Domains,” Nature 348:552-554 (1990); Clackson et al., “Making Antibody Fragments Using Phage Display Libraries,” Nature, 352:624-628 (1991); and Marks et al., “By-passing Immunization: Human Antibodies from V-gene Libraries Displayed on Phage,” J Mol Biol 222:581-597 (1991), which are hereby incorporated by reference in their entirety).

In addition to utilizing whole antibodies, the methods of the present invention encompass use of binding portions of such antibodies. Such binding portions include Fab fragments, F(ab)₂ fragments, Fab′ fragments, F(ab′)₂ fragments, Fd fragments, Fd′ fragments, Fv fragments, and minibodies, e.g., 61-residue subdomains of the antibody heavy-chain variable domain (Pessi et al., “A Designed Metal-binding Protein with a Novel Fold,” Nature 362:367-369 (1993), which is hereby incorporated by reference in its entirety). Domain antibodies (dAbs) (see, e.g., Holt et al., “Domain Antibodies: Proteins for Therapy,” Trends Biotechnol. 21:484-90 (2003), which is hereby incorporated by reference in its entirety) are also suitable for the methods of the present invention. These antibody fragments can be made by conventional procedures, such as proteolytic fragmentation procedures, as described in J. Goding, MONOCLONAL ANTIBODIES: PRINCIPLES AND PRACTICE 98-118 (1984), which is hereby incorporated by reference in its entirety.

Further, single chain antibodies are also suitable for the present invention (e.g., U.S. Pat. No. 5,476,786 to Huston and U.S. Pat. No. 5,132,405 to Huston & Oppermann; Huston et al., “Protein Engineering of Antibody Binding Sites: Recovery of Specific Activity in an Anti-digoxin Single-chain Fv Analogue Produced in Escherichia coli,” Proc. Nat'Acad. Sci. USA 85:5879-83 (1988); U.S. Pat. No. 4,946,778 to Ladner et al.; Bird et al., “Single-chain Antigen-binding Proteins,” Science 242:423-6 (1988); Ward et al., “Binding Activities of a Repertoire of Single Immunoglobulin Variable Domains Secreted from Escherichia coli,” Nature 341:544-6 (1989), each of which is hereby incorporated by reference in its entirety). Single chain antibodies are formed by linking the heavy and light immunoglobulin chain fragments of the Fv region via an amino acid bridge, resulting in a single chain polypeptide. The use of univalent antibodies is also embraced by the present invention.

Examples of antibodies against ApoA-1 are described in, e.g., Curtiss et al., “The Conformation of Apolipoprotein A-1 in High-density Lipoproteins Is Influenced by Core Lipid Composition and Particle Size: A Surface Plasmon Resonance Study,” Biochemistry 39:5712-5721 (2000); Bustos et al., “Monoclonal Antibodies to Human Apolipoproteins: Application to the Study of High Density Lipoprotein Subpopulations,” Clin. Chim. Acta 299:151-167 (2000); Marcel et al., “Lipid Peroxidation Changes the Expression of Specific Epitopes of Apolipoprotein A-1,” J. Biol. Chem. 264:19942-19950 (1989); McVicar et al., “Characteristics of Human Lipoproteins Isolated by Selected-affinity Immunosorption of Apolipoprotein A-1,” Proc. Nat'l Acad. Sci. USA 81:1356-1360 (1984); Miyazaki et al., “A New Sandwich Enzyme Immunoassay for Measurement of Plasma Pre-betal-HDL Levels,” J. Lipid Res. 41:2083-2088 (2000); Fielding et al., “Unique Epitope of Apolipoprotein A-1 Expressed in Pre-beta-1 High-density Lipoprotein and Its Role in the Catalyzed Efflux of Cellular Cholesterol,” Biochemistry 33:6981-6985 (1994), each of which is hereby incorporated by reference in its entirety.

Commercially available anti-human apoA-1 Mab 3A11-1A9 is available from Sigma-Aldrich (product WH0000335M1), and commercially available ELISA kits for detection of human ApoA-1 are available from Abnova (product H000035-AP21) and Mabtech (products 3710-1A-20 and 3710-1H-20), as well as other commercial suppliers.

Aptamers are a class of molecule that represents an alternative to antibodies in term of molecular recognition. Aptamers are peptides or oligonucleotide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold, “Systematic Evolution of Ligands by Exponential Enrichment: RNA Ligands to Bacteriophage T4 DNA Polymerase,” Science 249(4968):505-10 (1990), which is hereby incorporated by reference in its entirety. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena, “Aptamers: An Emerging Class of Molecules that Rival Antibodies in Diagnostics,” Clin. Chem. 45(9):1628-50 (1999), which is hereby incorporated by reference in its entirety.

Exemplary antibody mimics include, without limitation, those known as monobodies, which are derived from the tenth human fibronectin type III domain (¹⁰Fn3) (Koide et al., “The Fibronectin Type III Domain as a Scaffold for Novel Binding Proteins,” J. Mol. Biol. 284:1141-1151 (1998); Koide et al., “Probing Protein Conformational Changes in Living Cells by Using Designer Binding Proteins: Application to the Estrogen Receptor,” Proc. Nat'l Acad. Sci. USA 99:1253-1258 (2002), each of which is hereby incorporated by reference in its entirety); and those known as affibodies, which are derived from the stable alpha-helical bacterial receptor domain Z of staphylococcal protein A (Nord et al., “Binding Proteins Selected from Combinatorial Libraries of an alpha-helical Bacterial Receptor Domain,” Nature Biotechnol. 15(8):772-777 (1997), which is hereby incorporated by reference in its entirety). Variations in these antibody mimics can be created by substituting one or more domains of these polypeptides and then screening the modified monobodies or affibodies for apoA-1 binding specificity.

The aforementioned binding assays may involve the binding of the binding partner (i.e. antibody, antibody mimic, or aptamer) to a solid support. Solid supports which can be used in the practice of the invention include, without limitation, substrates such as nitrocellulose (e.g., in membrane or microtiter well form); polyvinylchloride (e.g., sheets or microtiter wells); polystyrene latex (e.g., beads or microtiter plates); polyvinylidine fluoride; diazotized paper; nylon membranes; activated beads, magnetically responsive beads, and the like.

The binding partners to be used in these assays may be labeled with a detectable molecule or substance, such as a fluorescent molecule, a radioactive molecule or any others labels known in the art. Labels are known in the art that generally provide (either directly or indirectly) a signal. As used herein, the term “labeled”, with regard to the binding partner, is intended to encompass direct labeling of the binding partner by coupling (i.e., physically linking) a detectable substance, such as a radioactive agent or a fluorophore (e.g., fluorescein isothiocyanate (FITC) or phycoerythrin (PE) or Indocyanine (Cy5)) to the binding partner, as well as indirect labeling of the binding partner by reactivity with a detectable substance. A binding partner of the invention may be labeled with a radioactive molecule by any method known in the art. For example radioactive molecules include, without limitation, I¹²³, I¹²⁴, In¹¹¹, Re¹⁸⁶, and Re¹⁸⁸.

In one embodiment, an ELISA method is used for measuring the apoA-1 concentration, wherein the wells of a microtiter plate are coated with a set of antibodies against apoA-1. The body fluid sample is then added to the coated wells. After a period of incubation sufficient to allow the formation of antibody-antigen complexes, the plate(s) can be washed to remove unbound moieties and a detectably labeled secondary binding molecule added. The secondary binding molecule is allowed to react with any captured apoA-1, the plate washed and the presence of the secondary binding molecule detected using methods well known in the art. As noted above, commercially available apoA-1 kits can be used to carry out the assay.

In another embodiment, the apoA-1 concentration of the body fluid sample may be performed using an array chip. Such an array technology allows a large number of experiments to be performed simultaneously on a single substrate, commonly known as a biochip when used for biological analytes. For example the binding partner for apoA-1 may be immobilized at the surface of the array chip. The body fluid sample obtained from the subject exposed to head trauma is then deposited on the array chip. After a period of incubation sufficient to allow the formation of binding partner-apoA-1 complexes, the array chip is then washed to remove unbound moieties, and thus allowing the isolation of apoA-1. In a second step, the measurement of apoA-1 concentration may be performed with a second binding partner specific for apoA-1. In a preferred embodiment, the second binding partner is labeled, thus allowing the formation of a set of “spots” (colored deposit) specific for apoA-1. For example, detection and quantification may be performed by analyzing the spots on the array chip with a specific detector.

Other emerging nanotechnologies may be used to quantify apoA-1, such as carbon nanotubules, quantum dots, silicon nanowires, and metal nanoparticles (Giljohann et al., “Drivers of Biodiagnostic Development,” Nature 426:461-464 (2009), which is hereby incorporated by reference in its entirety).

Regardless of the approach used for detecting the apoA-1 levels in the fluid sample, it is possible to quantifiably estimate the apoA-1 present in a body fluid sample of interest by comparing the measurements made using a particular assay to measurements made in preparing a calibration curve. The calibration curve is a plot of how the detected response, the so-called analytical signal, changes with the concentration of apoA-1, which is measured as, e.g., a series of standards across a range of concentrations near the expected apoA-1 concentrations in both normal and mTBI patients. Analyzing each of these standards using the chosen technique will produce a series of measurements, which can be plotted as a relationship between the detected analytic signal versus the apoA-1 concentration. When comparing the analytic signal obtained using the patient sample against the calibration curve, the user can interpolate to find the concentration of apoA-1 detected. This can be repeated several times, and an average response used for the patient in assessing when the patient has suffered an mTBI.

The determination of mTBI can be made based solely on the presence of or level of the apoA-1 biomarker, or it can be based on additional diagnostic markers or biomarkers. There are a number of additional biomarkers that can be used to assist in detecting or diagnosing mTBI following a head trauma. Biomarker S100B is a brain protein that can be used to predict the necessity of obtaining a head CT in a concussion patient. S100B is defined as a protein from the group consisting of the so-called “S100” proteins which, as their name implies, have the property of remaining in solution even at 100% saturation with ammonium sulphate at neutral pH (solubility 100%). They belong to the calcium-binding proteins, which are usually localized in cytoplasm. However, some S100 proteins, including S100B, also occur in the extracellular space. S100 proteins and their known properties, functions, and positive or negative effects in various pathological processes have been thoroughly studied, with particular emphasis those of the brain and central nervous system (Donato, “S100: A Multigenic Family of Calcium-Modulated Proteins of the EF-Hand Type With Intracellular and Extracellular Functional Roles,” Int. J. Biochem. Cell Biol. 33:637-668 (2001); Donato, “Functional Roles of S100 Proteins, Calcium-Binding Proteins of the EF-Hand Type,” Biochim. Biophys. Acta. 1450:191-231 (1999), which are hereby incorporated by reference in their entireties). For an individual testing positive for mTBI using apoA-1 levels, then it may be desirable to assess whether a CT scan is necessary. Detecting elevated S100B can be carried out according to the procedures described in Biberthaler et al., “Serum S-100B Concentration Provides Additional Information for the Indication of Computed Tomography in Patients After Minor Head Injury: A Prospective Multicenter Study,” Shock 25:446-453 (2006), which is hereby incorporated by reference in its entirety.

A subject who is conscious after exposure to a head trauma may be asymptomatic of any visible diagnostic markers of traumatic brain injury. Conversely, the subject may exhibit various diagnostic markers of brain injury and cognitive dysfunction.

Diagnostic markers that can be used to determine whether the subject that was exposed to head trauma has mTBI may include one or more than one of the following: memory loss; pupil dilation; convulsions; distorted facial features; fluid draining from nose, mouth, or ears; fracture in the skull or face; bruising of the face;

swelling at the site of injury; scalp wound; impaired hearing, smell, taste, or vision; inability to move one or more limbs; irritability; personality changes; unusual behavior; confusion; drowsiness; low breathing rate; drop in blood pressure; restlessness, clumsiness; lack of coordination, severe headache, slurred speech; stiff neck; and vomiting. A mild brain injury that occurs without loss of consciousness may leave a subject with merely a dazed feeling or confused state lasting a short time.

Brain injury symptoms frequently manifest themselves in combined deficits of attention, intention, working memory, and/or awareness. As used herein, attention refers to the cognitive function that provides the capacities for selection of internal or external stimuli and thoughts, supports the preparation of intended behaviors (e.g., speeds perceptual judgments and reaction times), and supports the maintenance of sustained cognition or motor behaviors (e.g., the focusing of attention). Intention, as used herein, refers to the mechanism of response failures (i.e., lack of behavioral interaction) which is not due to a perceptual loss (i.e., intention is the cognitive drive linking sensory-motor integration to behavior). Intention deficits include failure to move a body part despite intact motor pathways, awareness, and sensory processing as demonstrated by neurophysiological and neuropsychological evaluation. Another example of a patient's intention deficit is a failure to initiate action of any kind despite evidence of awareness or action produced by stimulation. Loss of intention is a disorder of cognitive function, as defined herein, and is a major division of the neuropsychological disorder of neglect, which may be present in many patients with cognitive loss following brain injury caused by a head trauma. Working memory, as used herein, refers to the fast memory process required for on-line storage and retrieval of information, including processes of holding incoming information in short-term memory before it can be converted into long-term memory and processes which support the retrieval of established long-term (episodic) memories. Deficits in awareness relate to impaired perceptual awareness, as described above. Clinical signs of these brain injuries also include profound hemi-spatial neglect, disorders of motor intention, disorders of impaired awareness of behavioral control, or apathy and cognitive slowing.

After a subject who was exposed to head trauma is subjected to the methods of the present invention to evaluate the presence of mTBI, the result of such will determine the course of treatment, if any, for the tested subject.

If the result of the method for mTBI detection is negative (i.e. apoA-1 levels are not decreased), then the subject does not have mTBI and, therefore, can resume normal daily activities fairly soon.

If, on the other hand, the result of the method for mTBI detection is positive (i.e. apoA-1 levels are decreased), then the subject should be treated for mTBI, including rest and refraining from all potentially dangerous activities that could inflict additional head trauma. ApoA-1 levels can be re-evaluated according to the method of the present invention at various time periods after the head trauma. For example, subsequent evaluations can occur approximately 24 hours, 48 hours, 72 hours, 96 hours, 120 hours, 144 hours, and/or one week after the trauma is inflicted, and anytime thereafter. This repeated course of testing, along with evaluation of diagnostic indicia of mTBI, will help evaluate the proper treatment, and whether the subject is ready to resume normal activities.

If the result of the method described herein indicates a borderline positive result (i.e., apoA-1 levels are only slightly decreased), then other diagnostic markers described herein can be used to assess the subject's potential need for treatment.

Exemplary methods of treatment include withholding physically strenuous activity or all activity for one week, or until apoA-1 levels return above a particular threshold value. In cases of mTBI, typical treatment includes restricting activity and minimizing risk of exposure to any additional head trauma.

The term “treating” as used herein, should be understood as partially or totally preventing, inhibiting, attenuating, ameliorating or reversing one or more symptoms or cause(s) of mTBI injury, as well as symptoms, diseases or complications accompanying mTBI injury.

A further aspect of the invention includes a kit that can be used to detect both apoA-1 levels and S100B levels in a single body fluid sample. The kit may include one or more binding partner reagents that bind specifically to apoA-1, and one or more binding partner reagents that bind specifically to S100B. The kit may further include one or more of the following: a solid surface, reagents for detecting a label, and instructions for carrying out detection of apoA-1 and S100B, as well as guidelines for identifying the existence of mTBI based on the results of using the kit on a body fluid sample and identifying whether a CT scan is warranted based on the results of using the kit on the body fluid sample.

### EXAMPLES

The following examples are provided to illustrate embodiments of the present invention but are by no means intended to limit its scope.

### Materials and Methods for Examples 1-3

Subject Enrollment: All studies received institutional review board approval. Informed consent was obtained prior to enrolling all subjects. All mTBI patients enrolled met a consensus definition of mTBI (American Congress of Rehabilitation Medicine Mild Traumatic Brain Injury Committee of the Head Injury Interdisciplinary Special Interest Group, “Definition of Mild Traumatic Brain Injury,” J. Head Trauma Rehabilitation 8:86-87 (1993), which is hereby incorporated by reference in its entirety).

For the proteomic studies, subjects with mTBI were selected from a large parent cohort accrued through the University of Rochester Medical Center Emergency Department (URMC ED). A total of 1910 patients were enrolled and a subset of 690 consented to having blood drawn. Three months after the initial URMC ED visit, post-concussive scores were determined by telephone interview using the Rivermead Post Concussion Questionnaire (RPCQ) (King et al., “The Rivermead Post Concussion Symptoms Questionnaire: A Measure of Symptoms Commonly Experienced after Head Injury and its Reliability,” J. Neurol. 242:587-592 (1995), which is hereby incorporated by reference in its entirety). Post-concussive symptom scores range from 0 (asymptomatic) to 64 (very symptomatic). For proteomic studies 3 symptomatic (mean PCS score=25, SD 10) and 3 asymptomatic (PCS score=0) subjects were selected from the cohort.

For both apo A-1 and S100B studies, mTBI and control subjects were enrolled as part of a multicenter study of patients presenting to the emergency department (ED) with clinically defined mTBI. Clinically defined mTBI includes at least one of the following: loss of consciousness (LOC)≦30 minutes; neuropsychological abnormality (e.g., any transient period of confusion, disorientation or impaired consciousness; and in children≦2 years old, either irritability, lethargy or vomiting post-injury); and neurological abnormality (e.g., seizure acutely following injury, hemiplegia, or diplopia). Uninjured subjects for S100B studies were enrolled from volunteers presenting to a clinical lab for routine outpatient blood draws.

Serum Collection and Handling: For apoA-1 studies in mTBI subjects, whole blood was collected by venipuncture into serum separator tubes and placed on ice. Samples were centrifuged to separate serum which was first frozen at −20° C. and then transferred to a −80° C. freezer. For all other subjects, blood was collected and processed similarly but was frozen at −80° C.

Subjects with extra cranial trauma only were enrolled from patients presenting to the ED who had an isolated extremity injury requiring an x-ray. Subjects were excluded if they had a blow to the head as part of their injury mechanism, or any symptoms of TBI.

Proteomics: Serum samples were thawed on ice, depleted of high abundance proteins using the ProteoExtract Albumin/IgG Removal Kit (Calbiochem, Gibbstown N.J.), and then concentrated with Vivaspin 500Max columns (Sartorius, Edgewood N.Y.). Proteins were separated by two-dimensional gel electrophoresis with isolectric focusing and SDS-PAGE for the first and second dimensions respectively. Proteins were stained and staining intensity of individual spots was measured and analyzed using PDQuest software (Bio-Rad, version 7.4.0). Spots identified as different between groups were cut from gels and analyzed by the Proteomics and Mass Spectrometry Core Facility at Cornell University for identification by MALDI-TOF/TOF mass spectroscopy.

Quantification of Proteins: ApoA-1 concentrations were measured in duplicate by ELISA (Mabtech; Cincinnati, Ohio). S100B concentrations were measured with an electrochemiluminescence immunoassay kit (Elecsys S100; Roche Diagnostics, Mannheim, Germany).

Statistics: Two-tailed Student's t-tests were used to compare spot intensities in the proteomic experiment. Because the results of these comparisons were considered putative subject to verification in a larger sample, multiple comparisons were not considered. Two-tailed Student's t-tests were used to determine differences between groups for apoA-1 and S100B serum concentrations. Receiver operator characteristic curve analysis was performed using GraphPad Prism 5 for Windows (La Jolla, Calif.). For all tests, a value of p<0.05 was considered significant.

### Example 1

### ApoA-1 as a Marker for mTBI

ApoA-1 was initially identified as a potential mTBI biomarker through a proteomic screen of sera collected from mTBI subjects. This screening study was designed to identify acute protein expression differences in mTBI subjects who subsequently develop post concussive symptoms relative to those who do not. Using mass spectroscopy, apoA-1 was identified as a differentially expressed protein in mTBI subjects. Verification of the relationship between acute apoA-1 concentrations and post concussive symptoms was then evaluated by measuring apoA-1 concentrations in a larger cohort.

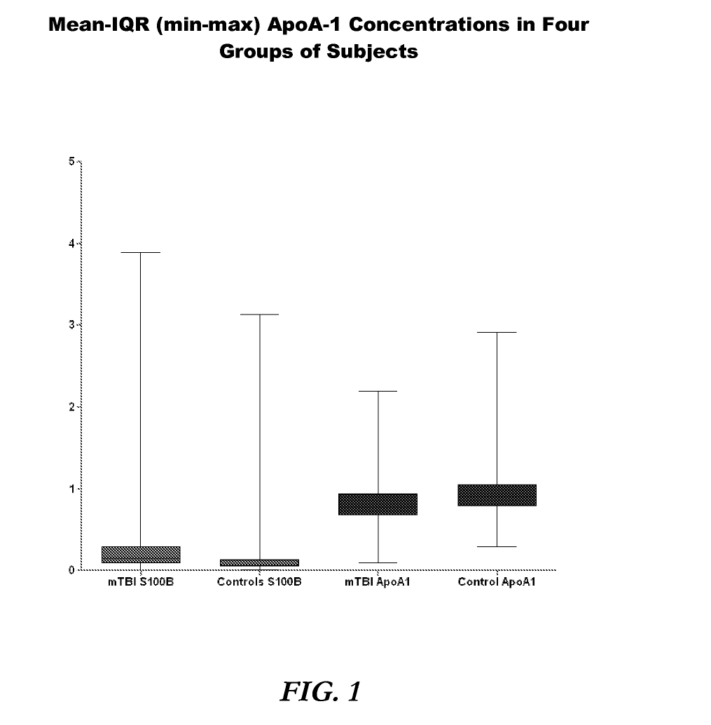

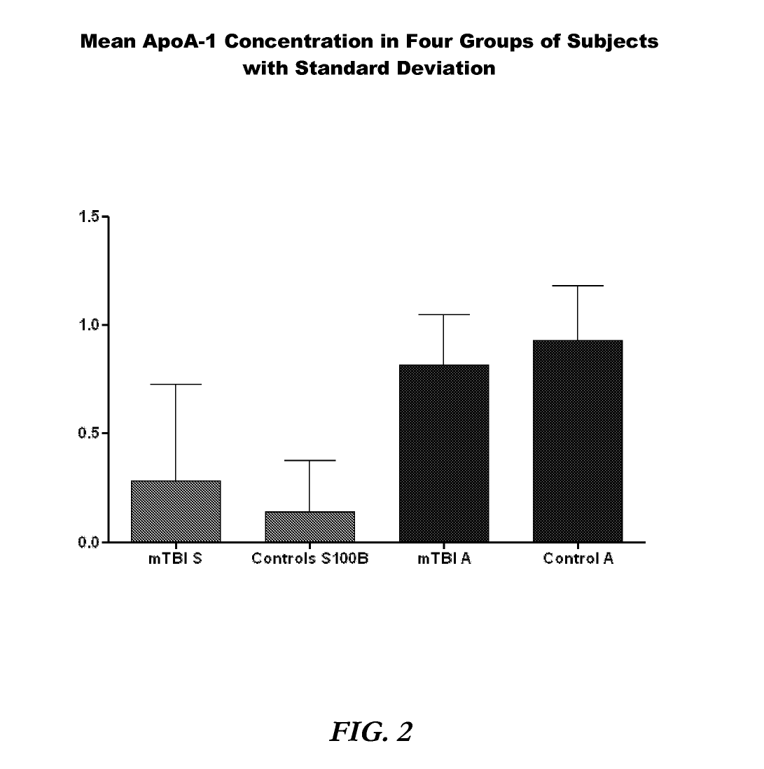

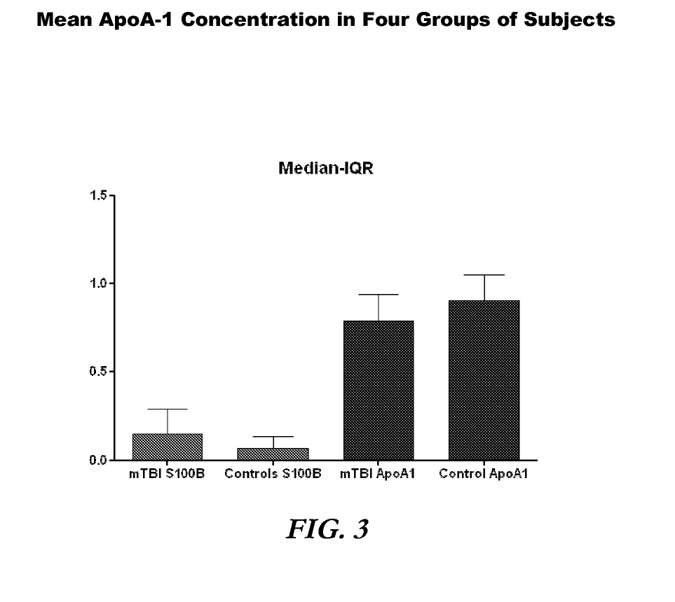

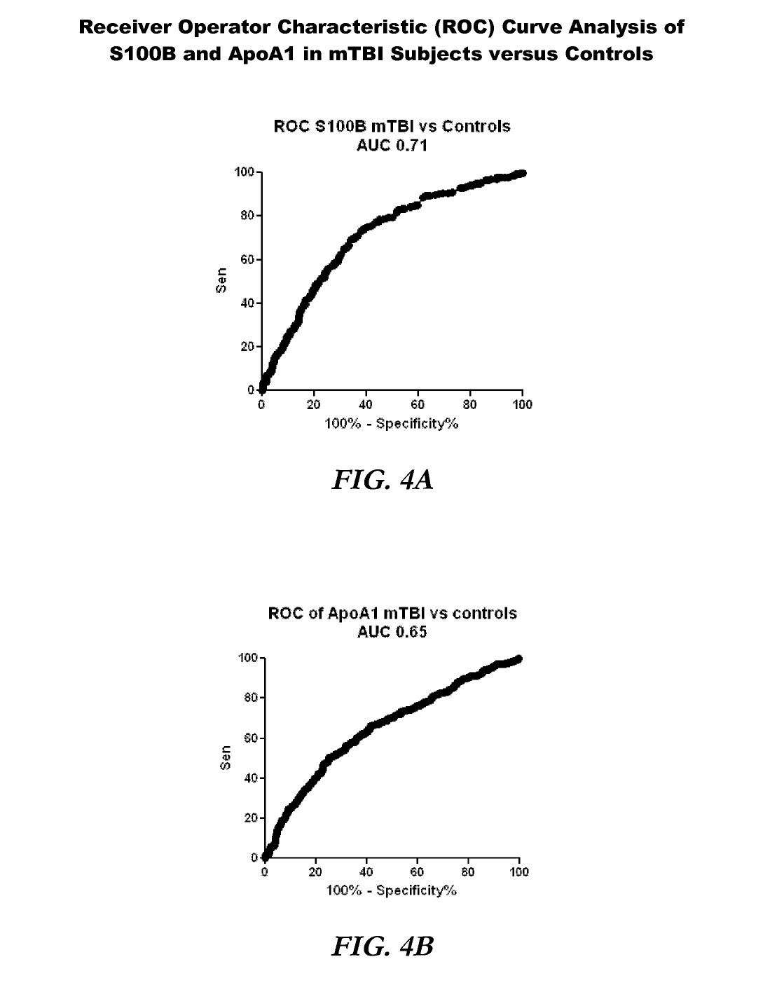

A total of 1254 subjects were enrolled (787 mTBI, 467 control). ApoA-1 concentrations were measured in 1241 of these subjects. ApoA-1 concentrations ranged from 0.10 to 2.20 mg/ml in mTBI and 0.30 to 2.92 mg/ml in control subjects (FIG. 1). Mean apoA-1 concentrations were 0.84 mg/ml (SD 0.25) and 0.95 mg/ml (SD 0.26) in mTBI and control subjects, respectively (FIG. 2). Median apoA-1 concentrations were 0.80 mg/ml and 0.92 mg/ml in mTBI and control subjects, respectively (FIG. 3). In a receiver operator characteristic analysis of the accuracy of apoA-1 to correctly classify subjects with mTBI compared with the gold standard of clinical diagnosis, the area under the curve was 0.65 (FIG. 4B).

### Example 2

### S100B Concentrations in mTBI Subjects

The only blood biomarker in clinical use with TBI is S100B, which is used widely in Europe but is not yet approved in the United States. Serum concentrations of this predominantly glial protein measured within 6 hours of injury are very sensitive for the identification of mTBI patients with traumatic injury detectable by cranial computed tomography scans (Biberthaler et al., “Serum S-100B Concentration Provides Additional Information for the Indication of Computed Tomography in Patients After Minor Head Injury: A Prospective Multicenter Study,” Shock 25:446-453 (2006), which is hereby incorporated by reference in its entirety). The accuracy of this test as a surrogate for clinical diagnosis is unclear, however.

A total of 1254 subjects were enrolled (787 mTBI, 467 control). S100B concentrations were measured in 1248 of these subjects. S100B concentrations ranged from 0 to 3.89 μg/L in mTBI and 0.01 to 3.13 μg/L in control subjects (FIG. 1). Mean S100B concentrations were 0.29 μg/L (SD 0.44) and 0.14 μg/L (SD 0.24) in mTBI and control subjects, respectively (FIG. 2). Median apoA-1 concentrations were 0.15 μg/L and 0.07 μg/L in mTBI and control subjects, respectively (FIG. 3). In a receiver operator characteristic analysis of the accuracy of S100B to correctly classify subjects with mTBI compared with the gold standard of clinical diagnosis, the area under the curve was 0.71 (FIG. 4A).

### Example 3

### Combining S100B and apoA-1 Concentrations Increases the Number of Subjects Correctly Classified

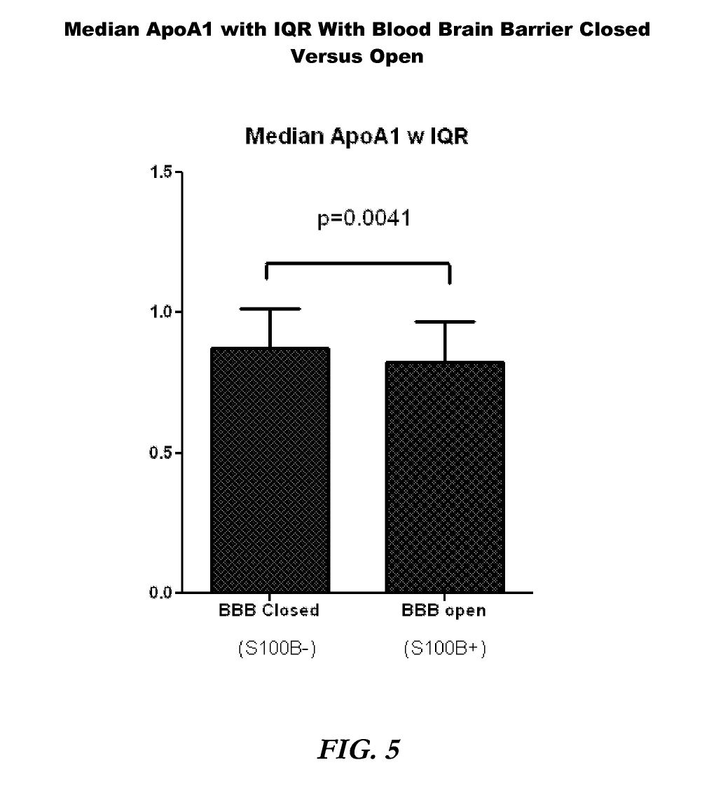

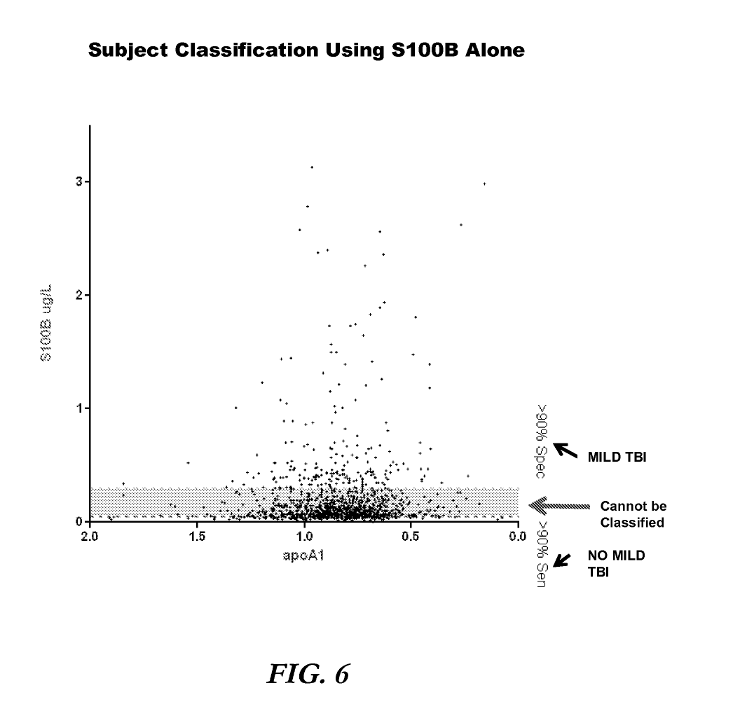

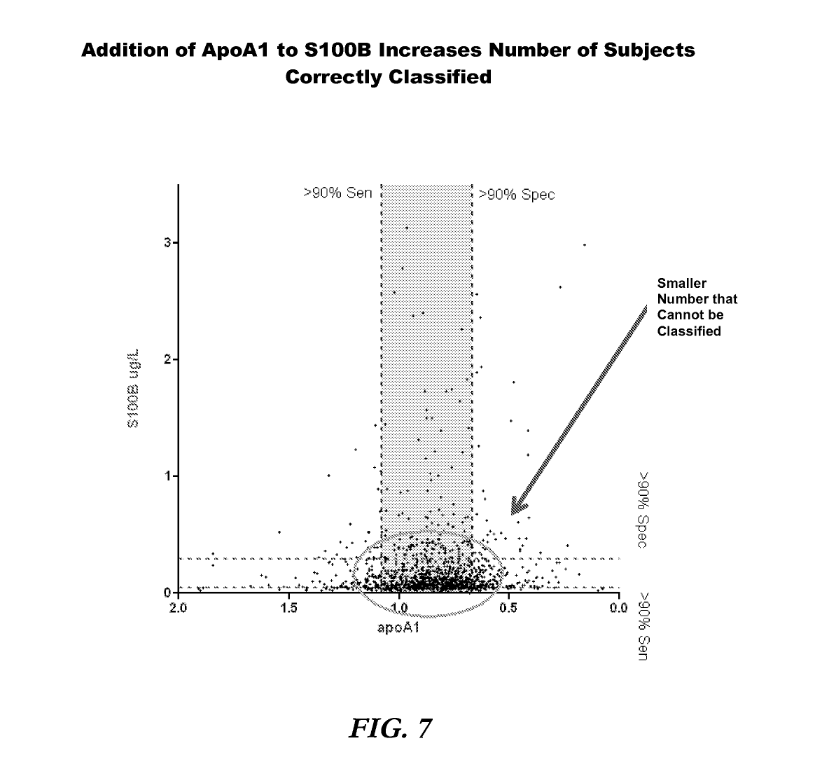

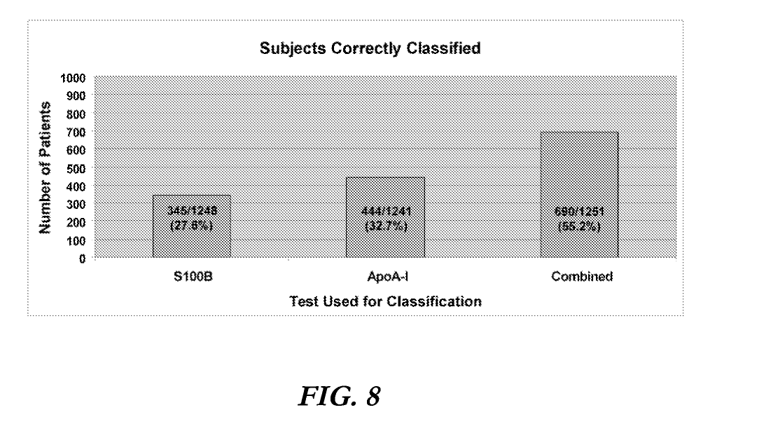

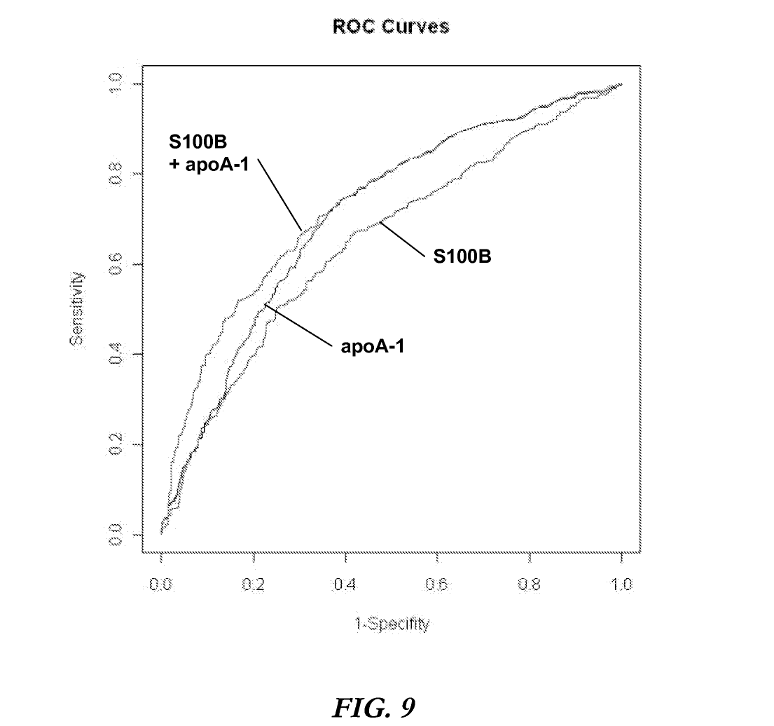

Elevated serum S100B concentrations are a surrogate for blood brain barrier function (Blyth et al., “Validation of Serum Markers for Blood Brain Barrier Disruption in Traumatic Brain Injury,” Journal of Neurotrauma 26:1497-1507 (2009), which is hereby incorporated by reference in its entirety). The blood brain barrier keeps brain derived proteins such as S100B from entering the peripheral circulation. The blood brain barrier can be referred to as “open” when S100B levels are elevated. ApoA-1 originates in the liver and bowel and is not made or produced in the brain. It is believed that apoA-1 might improve test accuracy by correctly classifying mTBI subjects with normal blood brain barrier function. ApoA-1 concentrations are significantly different between subjects with open vs. closed blood brain barrier (FIG. 5). Scatter plots of S100B vs. apoA-1 concentrations with the shaded areas representing subjects not correctly classified with mTBI at the 90% sensitivity and specificity cutoff for S100B and apoA-1 are shown in FIGS. 6 and 7, respectively. S100B, apoA-1 and the combined test correctly classify 345 of 1248 (27.6%), 444 of 1241 (32.7%) and 690/1251 subjects (5.2%) (FIG. 8), respectively. FIG. 9 shows the receiver operator characteristic curves for S100B (red or lowest), apoA-1 (black or middle), and the combined test (green or uppermost). The area under the receiver operator characteristic curve for the combined test is 0.74. The combined test is substantially better at diagnosing mTBI than S100B alone.

Although the invention has been described in detail for the purposes of illustration, it is understood that such detail is solely for that purpose, and variations can be made therein by those skilled in the art without departing from the spirit and scope of the invention which is defined by the following claims.

1-19. (canceled)

20. A method of diagnosing a subject for a mild traumatic brain injury, said method comprising:
obtaining a body fluid sample from a subject exposed to a head trauma; and
reacting the body fluid sample, or a portion thereof, with a reagent that binds specifically to apoA-1, and measuring the reagent/apoA-1 reaction product to determine an apoA-1 level in the obtained body fluid sample;
wherein a decreased apoA-1 level in the body fluid sample indicates that the subject has suffered a mild traumatic brain injury.

21. The method of claim 20, further comprising treating the subject for the mild traumatic brain injury.

22. (canceled)

23. The method of claim 20, wherein said obtaining is carried out within six hours of the head trauma.

24. The method of claim 20, wherein the subject is conscious at the time of said obtaining

25-27. (canceled)

28. The method of claim 20, wherein the decreased apoA-1 level is less than 0.68 mg/ml.

29. (canceled)

30. The method of claim 20, wherein the method is used with biomarkers other than apoA-1 to identify mTBI.

31. The method of claim 30, wherein the biomarker used is S100B.

32. The method of claim 20, wherein the method is used with other diagnostic markers to identify mTBI.

33. (canceled)

34. The method of claim 20, wherein said determining is carried out using an immunoassay.

35. The method of claim 20, wherein the method is used for treating the subject for mTBI based on said determining a decreased level of apoA-1 in the body fluid.

36. The method of claim 20, wherein the apoA-1 level is measured by a protein assay.

37-38. (canceled)

39. A method of treating an individual that has suffered a mild traumatic brain injury, said method comprising:
selecting a subject exposed to a head trauma; and
determining an apoA-1 level in a body fluid sample obtained from the selected subject, wherein a decreased apoA-1 level in the body fluid sample indicates that the subject has suffered a mild traumatic brain injury; and
treating the subject having suffered a mild traumatic brain injury as determined by said determining.

40-41. (canceled)

42. The method of claim 41, wherein said obtaining is carried out prior to said determining.

43. The method of claim 41, wherein the subject is conscious at the time of said obtaining.

44-46. (canceled)

47. The method of claim 39, wherein the decreased apoA-1 level is less than 0.68 mg/ml.

48. (canceled)

49. The method of claim 39, wherein the method is used with biomarkers other than apoA-1 to identify mTBI.

50. The method of claim 49, wherein the biomarker used is S100B.

51. The method of claim 39, wherein the method is used with other diagnostic markers to identify mTBI.

52. (canceled)

53. The method of claim 39, wherein said determining is carried out using an immunoassay.

54. The method of claim 39, wherein the apoA-1 level is measured by a protein assay.
